(19) United States
(12) Patent Application Publication
(10) Pub. No.: US 2017/0347905 A1
(43) Pub. Date: Dec. 7, 2017

(54) BRAIN ACTIVITY MONITORING, SUPPORTING MENTAL STATE DEVELOPMENT AND TRAINING

(71) Applicants: Graham Boulton, Windaroo (AU); Henry Boulton, Windaroo (AU)

(72) Inventors: Graham Boulton, Windaroo (AU); Henry Boulton, Windaroo (AU)

(21) Appl. No.: 15/603,501

(22) Filed: May 24, 2017

Related U.S. Application Data
(60) Provisional application No. 62/344,454, filed on Jun. 2, 2016.

(51) Int. Cl. A61B 5/04; A61B 5/0476; A61B 5/00

(52) U.S. Cl. CPC A61B 5/04012; A61B 5/742; A61B 5/0006; A61B 5/0476; A61B 5/7257

(57) ABSTRACT

Embodiments of the present invention are directed towards a system and method for determining an extent of concussion experienced by a user. The method includes receiving, by a computing device, electroencephalogram (EEG) data related to the user from an external device associated with the user. The method further includes determining, by the computing device, the extent of concussion based on the EEG data, displaying, by the computing device data related to the extent of concussion. The method further includes determining the extent of concussion based on the EEG data related to brain activities of the user before the user is concussed, during the time of concussion, and after the user is concussed. Embodiments of the present invention further helps in developing technical skills in the users during training apart from helping them realize or visualize the concussion and impact of the concussion during training or while participating in athletic activities.

### CROSS REFERENCE TO RELATED APPLICATION

The present application claims the priority benefits of U.S. Provisional Application No. 62/344,454, filed on Jun. 2, 2016, titled “BRAIN ACTIVITY MONITORING, SUPPORTING MENTAL STATE DEVELOPMENT AND TRAINING”, which is incorporated herein by reference in its entirety.

### FIELD OF THE INVENTION

The present application generally relates to EEG-based brain activity monitoring, and in particular, it relates to a method and system for determining an extent of concussion experienced by a user, such as for example, a player or an athlete involved in a specific sports. The present invention further relates to a system that can facilitate in developing technical skills among the users/players through effective training.

### BACKGROUND

In a field of performance psychology, optimal achievement of a particular task results from a presence of a psychological state. The psychological state comprises optimal values measured from psychological signals. Putting a golf ball, scoring a boundary, shooting a basketball, or throwing darts are all examples of activities and their successful completion is linked to the presence of an optimal achievement state concurrent with the psychological state of a player involved in respective games.

Further, knowledge of concussion (G-Force) hitting the player's brain while playing is vital. There is a mounting concern among number of users, specifically the athletes/players that often get concussed during a game play. Thus, the players/athletes getting concussed during their game play could be a big concern for parents, sporting bodies, and insurance companies.

Further, such concussive and/or sub-concussive hits onto the brain experienced by the players/athletes have been correlated with long term chronic and deleterious effects onto their brain. Cumulative effects of the concussive and sub-concussive that hits brains of the players/athletes may result in various psychiatric disorders and/or long term memory loss in them. Further, repetitive concussion in the players/athletes especially children and adolescence (low age group players in general) may impact their brain development process.

Despite several solutions proposed in the past to mitigate affect of concussion, it has remained a significant problem. Some research have demonstrated that some of existing head protection devices as a solution to overcome affects of the concussion may actually increase head accelerations and exacerbate the risk of concussions in the users. In order to properly manage concussion among the athletes/players, it may be necessary to remove the athletes suspected of sustaining concussion during the game play. The athletes who are not removed, or who are returned to the game play prematurely after sustaining a concussive hit may be at an increased risk of a catastrophic outcome known as ‘second impact syndrome’. The second concussion may occur in minutes, days, or weeks after the prior concussion. Even mild grades of concussions may lead to disability or death.

In addition, a coherence level of psychological state of workers and an operator may be vital to achieve desired productivity results in a work place, thus there is a need for a system that would facilitate with analyzing the electrical activities of the brain both at a prior state before occurrence of the concussion and at a post state after the occurrence of the concussion.

Conventionally, methods for studying psychological state has required analysis of the electrical activities of the brain. Conventional methods uses electrodes adhered to a user's scalp in combination with associated electronics such as amplifiers, filters, converters, integrators or the like, and then a display of an electroencephalogram (“EEG”) was studied for a given time period depicting the electrical activities of the brain at the various electrode sites. In general, EEG signals have been studied in an effort to determine relationships between frequencies of electrical activities or neural discharge patterns of the brain and corresponding psychological state. Such monitoring of the user's EEG and providing feedback information to the user as a function of the EEG may serve to enable a user's ability to reach a target psychological state and thus enhance the user's performance level. However, such conventional solutions are not fit for the users involved in various games/sports who may at any instant be hit by the concussion (real time monitoring).

In the light of above described background art, there is a need for a system and method that would facilitate in determining an extent of concussion experienced by the user in real time, such as the player or the athlete involved in a specific sport. The present invention further helps in developing technical skills among the users/players through effective training.

### BRIEF SUMMARY

An object of the embodiments of the present invention described herein is to provide a method and system for determining an extent of concussion experienced by a user.

Another objective of the present invention is to provide a method and system that would develop cognitive skills as well as technical skills among the users to make them more focused and competent for the activities (such as games) they are participating in.

According to an aspect of the present invention there is provided a method for determining an extent of concussion experienced by a user. The method comprising receiving, by a computing device, a plurality of electroencephalogram (EEG) data related to the user via an external device associated with the user; determining, by the computing device, the extent of concussion based on the plurality of EEG data received from the user via the external device; and displaying, by the computing device, a set of data related to the extent of concussion.

According to another aspect of the present invention there is provided a computing device for determining an extent of concussion experienced by a user, the computing device comprising a memory; a transceiver to receive a plurality of electroencephalogram (EEG) data related to the user via an external device associated with the user; a processor coupled to the memory; a G-force analyzer communicatively coupled to the processor, the processor configured to: determine the extent of concussion based on the plurality of EEG data; and display a set of data related to the extent of concussion.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

This invention is illustrated in the accompanying drawings, throughout which like reference letters indicate corresponding parts in the various figures. The embodiments herein will be better understood from the following description with reference to the drawings, in which:

FIG. 1 is an example scenario illustrating a system for determining an extent of concussion experienced by a user, according to an embodiment of the present invention;

FIG. 2 illustrates a block diagram of a computing device used for determining an extent of concussion experienced by the user, according to an embodiment of the present invention;

FIGS. 3A-3B illustrates a step by step process for determining an extent of concussion experienced by the user, according to an embodiment of the present invention;

FIG. 4 is an example scenario illustrating a method for determining a performance metric of the user among a group of users, according to an embodiment of the present invention;

FIG. 5 is an example illustrating a method for displaying data related to the performance metric of the user, according to an embodiment of the present invention;

FIG. 6 is an example illustrating a method for displaying data related to an extent of concussion experienced by the user, according to an embodiment of the present invention;

FIG. 7 is a flow diagram illustrating a method for determining the extent of concussion experienced by the user, according to an embodiment of the present invention;

FIG. 8 is a flow diagram illustrating a method for determining the performance metric associated with the user, according to an embodiment of the present invention;

FIG. 9 shows a schematic diagram for training the user, according to an exemplary embodiment of the present invention;

FIG. 10 shows an exemplary visual screen displayed on the computing device that helps the user visualize the concussion, and help in developing the technical skills during the training; and

FIG. 11 illustrates an example computing environment adapted for implementation of the method and system adapted for determining the extent of concussion experienced by the user, according to embodiments of the present invention.

### DETAILED DESCRIPTION

Some embodiments, illustrating its features, will now be discussed in detail. The words “comprising”, “having”, “containing” and “including”, “comprises of” and other forms thereof, are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise. Further, in the context of the present invention the terms “games”, “sports”, and other forms thereof are synonymously used throughout the description. Further, in the context of the present invention the terms “players”, “users”, “athletes” and other forms thereof are synonymously used throughout the description to denote a user.

The embodiments of the present invention described herein discloses a method for determining an extent of concussion experienced by a user. The method comprising receiving, by a computing device, a plurality of electroencephalogram (EEG) data related to the user via an external device associated with the user; determining, by the computing device, the extent of concussion based on the plurality of EEG data received from the user via the external device; and displaying, by the computing device, a set of data related to the extent of concussion.

Unlike the conventional systems and methods, the proposed method includes determining the extent of concussion that hits the user brain based on EEG data related to brain activities of the user before the brain is concussed, determining the extent of concussion based on the EEG data related to brain activities of the user during time of concussion, and determining the extent of concussion based on the EEG data related to brain activities of the user after the brain is concussed. Thus the proposed method provides a benchmark to ascertain the seriousness of the concussion for a physician. Further, the proposed method includes a dual parallel fast Fourier transformation (FFT) mechanism for eliminating interference noise from the digital EEG data so that a Mushin state of the user can be determined.

In an embodiment, the proposed method further includes determining brain state of the user by comparing the analog EEG data of the user wearing or using an external device with a metric. In an embodiment, the method further includes determining a performance metric of the user by analyzing the brain state of the user over a period of time.

Unlike the conventional systems and methods, the proposed method provides the performance metric of a user among a group of users. So the user having a poor performance metric among the group of users get a warning to improve his brain state. Further, the method provides dynamic conversion of the EEG data received from the external device to a color value or an audio visual representation based on a characteristic of the EEG data. Unlike the conventional methods and systems, the proposed method provides in-situ neurologic and physiologic monitoring of the user. Further, the proposed method provides the performance metric of a user who is associated to a different group of users. For example, the proposed method receives and analyzes the EEG data from different groups where one group competes with other groups in a group game, for example a football game. Further, the proposed method includes a voice guided blueprint that analyzes the root cause of the indifference among the users based on the performance matrices and also guides the users through drills to remedy the indifferences. Further, the proposed method provides high fidelity human health and performance data without interfering with the user's athletic activity or routine.

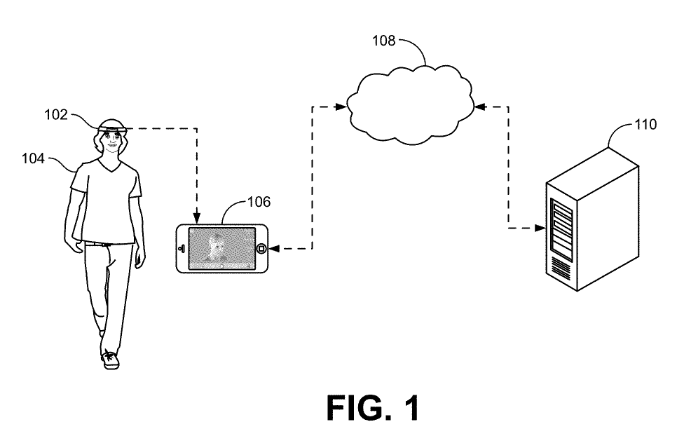

Further, the following detailed description is intended to provide example implementations to one of ordinary skill in the art, and is not intended to limit the invention, as one of ordinary skill in the art will understand that variations can be substituted that are within the scope of the invention as described. Reference will now be made to the accompanying drawings: FIGS. 1-11.

Referring to FIG. 1 is an example scenario illustrating a system for determining an extent of concussion experienced by a user. The system 100 includes an external device 102, the user 104, a computing device 106, a communication network 108, and a server 110. The user 104 preferably includes without any limitation a player, a coach or a physician. The external device 102 is associated with the user 104 and is configured to capture electroencephalogram (EEG) data of the user 104. Examples of the external device 102 may include, but are not limited to a headband, a headphone, a wearable gear, an electrode placed on the scalp, a bracelet, or any other user wearable accessories. In an embodiment, the external device 102 may be attached to the scalp of the user 104 by various means, including, but not limited to, adhesives, headbands, visors, caps, hats, helmets, clamps, clips and earring studs. In a preferred embodiment, the means of attachment of the external device 102 to the head of the user 104 is a cap. In an embodiment, the server 110 includes a cloud platform.

According to the embodiment, the external device 102 may be configured to be worn during an athletic activity, as well as any other activity such as during training. “Athletic activity” as used herein, does not refer to a specific sport or athletic event, but instead refers to any activity in which the wearer of the external device 102 may potentially sustain the concussion and/or desires to quantify and track their health and performance metrics.

In an embodiment, the external device 102 may be equipped with at least one of accelerometers, magnetic sensors, reed relays, RFID, photo-sensors, pressure sensor, piezoelectric sensors, tilt sensors, microphones, radio antennas, proximity sensor, tactile sensor, thermocouples, electro-chemical sensors, gyroscopes, humidity sensors, acoustic sensor, vibration sensors, temperature sensors, hydration sensors, moisture sensor, tilt sensor, inertial measurement unit sensor, compass, inclinometer, altimeter, GPS, pulse-oximeter sensors, EEGs, EKGs, voltmeters, ammeters, capacitance meter, inductance meter, resistance meter, LCR meter, or any combination thereof.

According to the embodiment, the external device 102 may capture EEG data from different groups where one group competes with other groups in a group game. According to the embodiments, the proposed method provides in-situ neurologic and physiologic monitoring of the user 104. In clinical contexts, EEG refers to the recording of the brain's spontaneous electrical activity over a period of time. In an embodiment, the EEG data includes neural oscillations and event-related potentials. Further, in clinical context, event-related potentials (ERPs) refer to averaged EEG responses that are time-locked to more complex processing of brain stimuli.

According to the embodiment, the captured EEG data includes concussion (G-Force) data. For example, the G-force on an object sitting on the Earth's surface is caused by mechanical force exerted in the upward direction by the ground, keeping the object from going into free-fall. The upward contact-force from the ground ensures that the object at rest on the earth's surface is accelerating relative to the free-fall condition.

In the embodiment, the concussions may occur as a result of at least one of repeated head accelerations, single large head acceleration, or a combination of both. According to the embodiment, the external device 102 is equipped with one or more large soft woven sensors reliable and capable of capturing the EEG data from the user 104 in real time. Unlike the conventional systems, the proposed solution uses the one or more soft woven sensors that ensure the injury is not exacerbated by the monitoring equipment while inside the helmet or the like. The woven sensor provide wide surface that is artifact tolerant, hence can be used in-situ. The woven sensors may be configured in such a way that any acceleration, rotation, displacement, alteration of position, etc. of the wearer at the point of attachment are properly mimicked.

In context of the present invention, the computing device 106 refers to a handheld electronic device that can be used to communicate over the communication network 108. Examples of the computing device 106 include, but are not limited to a cell phone, a smart phone, a cellular phone, a cellular mobile phone, a personal digital assistant (PDA), a wireless email terminal, a navigation terminal, a directional finder, a route planner, a laptop, and a tablet computer. Examples of types of the communication network 108 include, but are not limited to a local area network, a wide area network, a radio network, a virtual private network, an Internet area network, a metropolitan area network, a satellite network, Wi-Fi, Bluetooth Low energy, a wireless network, a cloud network, and a telecommunication network. Examples of the telecommunication network include, but are not be limited to a global system for mobile communication (GSM) network, a general packet radio service (GPRS) network, third Generation Partnership Project (3GPP), an enhanced data GSM environment (EDGE), 4G, LTE, LTE-A, and a Universal Mobile Telecommunications System (UMTS).

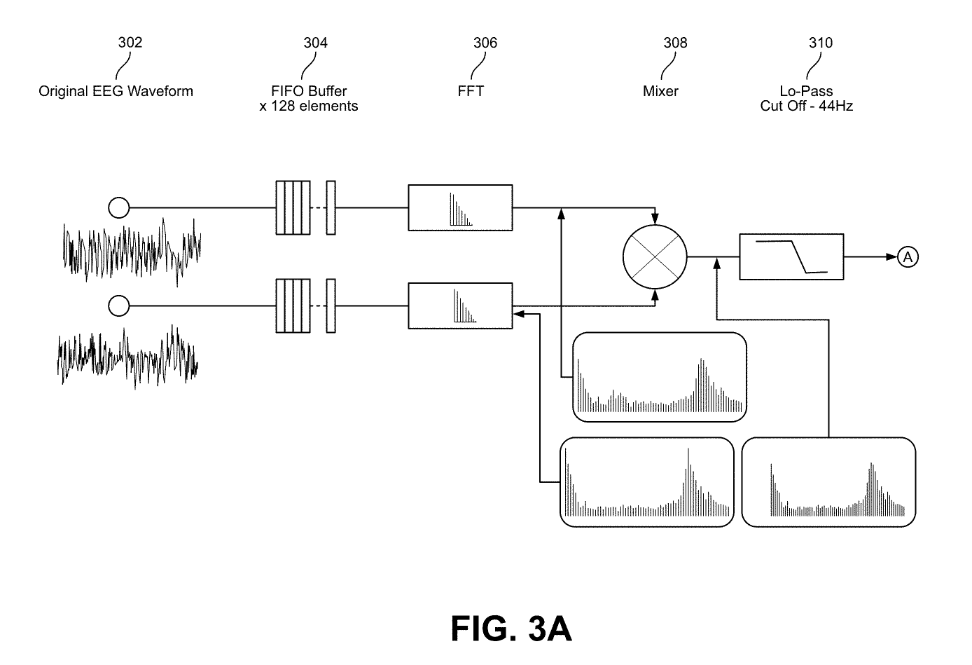

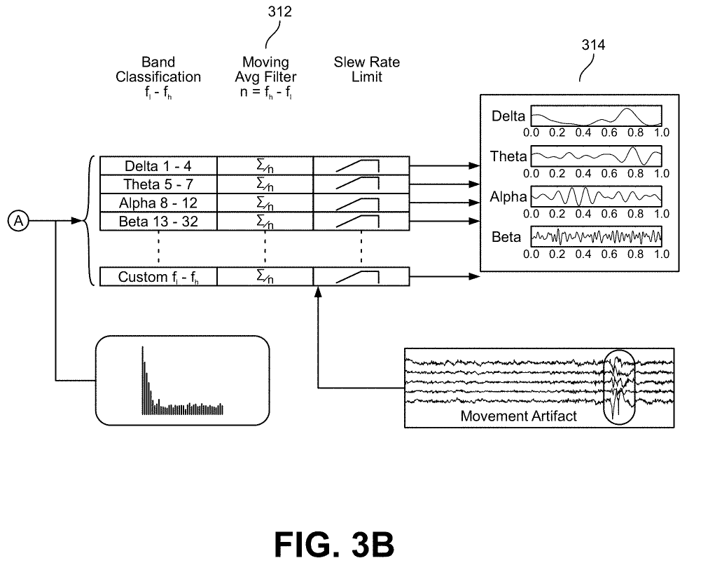

In the context of present invention, the external device 102 sends the captured EEG data of the user 104 to the computing device 106. According to the embodiment, the captured EEG data is analogue data. The computing device 106 is configured to convert the received analogue data from the external device 102 to a digital data. In an embodiment, the computing device 106 includes an analogue to digital converter that converts analog EEG data to digital EEG data. Further, the computing device 106 is configured to eliminate interference noise from the digital EEG data. The computing device 106 eliminates the interference noise from the digital EEG data using dual parallel fast Fourier transformation (FFT) mechanism. The dual parallel fast Fourier transformation (FFT) process nullifies the interference that occurs while processing EEG data that is collected from different users though the external device 102 associated with them. After eliminating the interference noise the computing device 106 forwards the EEG data optionally to the server 110, in case the processing of data has to be carried out by server 110. However, according to the described embodiment, the computing device 106 is capable of carrying out all the processing activities alone or in conjunction with the server 110. Unlike the conventional systems, the proposed method proposes an effective solution to eliminating interference noise from the digital EEG data using the dual parallel fast Fourier transformation (FFT) mechanism that ensures the proposed method can be used in-situ. The dual parallel fast Fourier transformation (FFT) mechanism is described in conjunction with FIGS. 3A and 3B below.

Further, the computing device 106 is configured to determine the extent of concussion based on the digital EEG data after eliminating the interference noise from the digital EEG data. According to the embodiment, the computing device 106 is configured to determine the extent of concussion based on a comparison of digital EEG data received from the user via the external device 102 with a metric stored therein. The data of the metric is shown in Table 1 below. According to the embodiment, the computing device 106 is configured to determine the extent of concussion based on EEG data related to brain activities of the user 104 before the brain is concussed, during the concussion and after the brain is concussed.

According to some embodiments, the computing device 106 may be configured to record the biometric data of the user 104 prior to, during, or subsequent to engagement in an athletic activity. The recorded biometric data may be used to analyze the user's health and performance, and may also be used to track the same health and performance metrics over a period of time. This may aid in the recognition of the concussion, which may be too subtle or difficult to detect without performance metric tracking. Recorded biometric data may provide for an objective means of tracking health and performance trends and recognizing health events. A potential concussion may trigger the computing device 106 to report the potential concussion associated to the user 104.

According to the embodiment, the computing device 106 is configured to determine brain state of the user 104 by comparing the analog EEG data of the person 104 with associated external device 102 with a metric. An example data of the metric is shown in Table 1 below.

TABLE 1

| | Frequency | |
|---|---|---|
| | range | |
| Name | (Hz) | Brain State of a person |
| Delta | 0-4 | Sleep, unconscious processing |
| Theta | 4-7 or 4-8 | Deeply relaxed, inwardly focused |
| Alpha | 8-12 or 8-13 | Very relaxed, passive attention |
| Beta | >13 | External attention |
| SMR Beta | 12-15 | Relaxed, external attention |
| Mid Beta | 15-18 | Active, external attention |
| High Beta | 18-35 | Anxiety, external attention |
| Gamma | >30 or >35 | Peak performance states or a |
| | | consolidation frequency |

As shown in example Table 1, if the analog EEG data of the user 104 is 3 Hz, then the computing device 106 is configured to determine brain state as unconscious. Similarly, if the analog EEG data of the user 104 is 20 Hz, then the computing device 106 is configured to determine brain state as an anxiety state.

Further, according to the embodiment, the computing device 106 may record acceleration, rotation, displacement, and/or geographic position information of the user 104. In some embodiments, the computing device 106 may be used to detect head, body, or extremity tilt and direction. The recorded information may be used to determine the possible occurrence of the concussion.

According to the embodiment, the computing device 106 is configured to calculate the severity level for a single impact, a plurality, or an entirety of the one or more impacts. In an example, the computing device 106 is configured to calculate the severity level by comparing the EEG data along with sensor data (e.g., vibration data, acceleration data, rotational data, or a combination thereof) that is collected from difference sensors against the threshold data. For example, the computing device 106 is configured to determine if the user 104 sustained an impact consisting of a linear impact component (e.g., x number of g-forces (g's)) or a rotational movement or angular acceleration component (e.g., x number of radians/s2) and determines the severity level based on those values compared against the impact threshold criteria that the user 104 has sustained the concussion previously.

Further, the computing device 106 is configured to display data related to the extent of concussion. In an embodiment, the computing device 106 is configured to display data related to the performance metric of the person 104. In an embodiment, the data related to the extent of concussion is displayed in a visual form. In another embodiment, the data related to the extent of concussion is converted to a color value or audio visual representation based on a characteristic of the EEG data. Examples of visual form include, but are not limited to emoticons, graphical character, chart, avatars, and graphical reports. In an embodiment, the visual form includes quantitative analysis of analyzed EEG data. In an example, the quantitative analysis of analyzed EEG data refers to degree of similarity of the analyzed EEG data with the range of frequencies mentioned in Table 1, shown above. In another example, the quantitative analysis of analyzed EEG data refers to percentage calculation. In an embodiment, the analyzed EEG data provide a benchmark to ascertain the seriousness of the knock for a physician.

According to the embodiment, the computing device 106 is configured to monitor the one or more impacts sustained by the user 104 over a period of time (e.g., duration of the team/group activity). In particular, the user 104 that may have sustained a number of impacts over the period of time may be at the same level of risk to short-term or long-term injury as another user that may have sustained a single severe impact even if the impacts over the period of time are minimal or moderate in severity. In addition, the user that has sustained a severe impact (e.g., a concussion) may be more susceptible to sustain another concussion from one or more impacts, particularly if the time between such one or more impacts is minimal.

According to the embodiment, the computing device 106 is configured to generate a recommendation for a level of participation of the user 104, the one or more other users, or a combination thereof in a team/group activity based on the one or more impacts, a severity level of the one or more impacts, or a combination thereof.

According to another embodiment, the computing device 106 is configured to “predict” a level of risk of short-term or long-term injury for the user 104 based on the one or more impacts sustained by the user 104 over the period of time and can then generate the recommendation for the level of participation for the user 104 accordingly. For example, based on the impacts sustained by the user, the computing device 106 may be configured to recommend not to continue to participate in the activity for the duration of the activity, the duration of the season (e.g., if the user has already sustained a number of severe impacts over the course of the season or his career depending on the particular circumstances).

In yet another embodiment, the computing device 106 is configured to recommend to a coach monitoring the user/player 104 stating the user 104 is at a moderate to high level of risk of injury and therefore should be carefully watched for a period of time (e.g., the next few plays) or be immediately examined by a medical personnel (e.g., a clinician, a trainer, etc.) for possible signs of a mild traumatic brain injury or a concussion upon sustaining one or more impacts. Further, the computing device 106 may be configured to recommend that the user 104 is safe to participate or to continue participating in the activity despite having sustained one or more impacts (e.g., one or more impacts of minimal severity).

In one embodiment, the computing device 106 is further configured to determine a substitute for the user 104 participating in the team activity based on the one or more impacts. For example, if the computing device 106 is configured to detect if the user 104 has sustained a number of impacts throughout the course of the activity (e.g., a football game), the system can suggest a substitute user that can replace the user 104, such substitution may be for example, during the particular activity or for a period of time (e.g., the next scheduled activity). According to the embodiment, the computing device 106 is configured to determine the one or more substitutions based on the premise that there should be no severely impacted users (may be players) participating in the activity at any given time and that the number of cautionary users participating in the activity should be minimized as much as possible. Therefore, the computing device 106 is configured to possibly determine the one or more substitutions so that the one or more users participating in the activity are safe and fit to participate and/or to continue participating in the activities (e.g., green users).

In an embodiment, the computation device 106 further includes an alert module. The alert module is configured to send notifications to an intended user. Examples of the notification may include, but are not limited to an email, text message, short message service (SMS), video clip, audio response, Interactive voice response (IVR), and a popup application.

In an embodiment, the computation device 106 further includes a performance mapping module. The performance mapping module compares the brain state of the user 104 with brain state of another user in the group of users. Further, the performance mapping module determines a performance metric of each user from the group and provides feedback to each user from the group on improvising performance of each user of the group to achieve the objective of the group to which the user belongs.

In another embodiment, the performance mapping module determines brain state of the user 104 and provides feedback to the user 104 to attain a Mushin state. In context of the present invention, the Mushin state refers to a state of mind where discursive thought and judgment are absent and the user 104 is driven towards the objective of the group. In an example, consider a golf match where a golfer, a caddie and a coach are present. Each having the external device 102 placed on their scalp. The external device 102 collects EEG data from the golfer, the caddie and the coach and sends the collected EEG data to the corresponding computing devices associated with the golfer, caddie and coach. Consider a scenario where the computing device 106 that is associated with the golfer receives the EEG data. The computing device 106 is configured to determine the brain state of the golfer by comparing the EEG data with the metric. If it is determined that the brain state of the golfer is not in the range of the Mushin state frequency, then the computing device 106 provides a training methodology to improve the brain state of the golfer. After the training, the golfer attains the Mushin state.

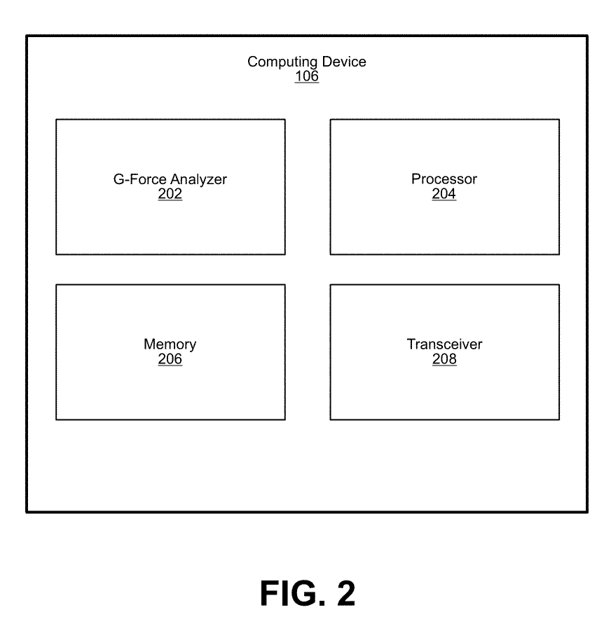

Referring to the FIG. 2 illustrates a block diagram of the computing device 106 for determining the extent of concussion experienced by the user 104. In an embodiment, the computing device 106 includes a G-force analyzer 202, a processor 204, a memory 206, and a transceiver 208. The G-force analyzer 202 communicatively linked to the processor 204 and configured to determine the extent of concussion based on the EEG data. Further, the G-force analyzer 202 is configured to display data related to the extent of concussion onto the computing device 106.

The processor 204 may be, for example, a hardware unit, an apparatus, a Central Processing Unit (CPU), a Graphics Processing Unit (GPU)) communicatively coupled to the memory 206 (e.g., a volatile memory and/or a non-volatile memory). The memory includes storage locations configured to be addressable through the processor 204. Further, the memory 206 may include non-volatile storage elements. Examples of such non-volatile storage elements may include magnetic hard discs, optical discs, floppy discs, flash memories, or forms of electrically programmable memories (EPROM) or electrically erasable and programmable (EEPROM) memories. In addition, the memory 206 may, in some examples, be considered a non-transitory storage medium. The term “non-transitory” may indicate that the storage medium is not embodied in a carrier wave or a propagated signal. However, the term “non-transitory” should not be interpreted that the memory 206 is non-movable. In some examples, the memory 206 can be configured to store larger amounts of information than the memory. In certain examples, a non-transitory storage medium may store data that can, over time, change (e.g., in Random Access Memory (RAM) or cache). The transceiver 208 can be configured for communicating internally between the units and externally with the networks. Further, the transceiver 208 is configured to receive electroencephalogram (EEG) data from the user 104 transmitted by the external device 102 associated with the user 104. According to some other embodiment, the transceiver 208 may also be configured to receive data from other sensors (for example, tilt sensor, accelerometer and so on) worn by the user 104 during training, for example, sensors present on the body locations such as pelvic area, thorax region, and sensor on the club held by the user and so on. Such data from other sensors may be analyzed and processed by the processor 204 based on set of instructions stored in the memory 206 to help meet the objectives of the training. Analysis and processing of such data from other sensors apart from the EEG data received from the external device 102 may be needed during training to make the user/player 104 realize about the actual body positions that they should have while participating in activities (for example, training required for the game of Golf), while they are getting trained to achieve mushin state.

Referring to FIGS. 3A-3B that that illustrate step by step process for determining the extent of concussion experienced by the user 104. At step 302, the computing device 106 is configured to receive the EEG data from the user 104 via the external device 102 worn by the user 104. As shown, at step 304, the computing device 106 is configured to store the EEG data in a FIFO (first in first out) buffer on a first-in, first-out basis. The FIFO buffers are used to manage asynchronous EEG data collected from the external device 102 at different time. The storage structure of FIFO buffer is typically an array of contiguous memory. The EEG data is written to the “head” of the buffer and read from the “tail”. When the head or tail reaches the end of the memory array, it wraps around to the beginning. If the tail runs in to the head, the buffer is empty.

As shown, at step 306, the computing device 106 is configured to process the EEG data through dual parallel fast Fourier transformation (FFT) mechanism. The FFT is a fast implementation of the Discrete Fourier Transform (DFT) wherein the EEG data are converted from time domain EEG data into its corresponding frequency domain EEG data. The dual parallel fast Fourier transformation (FFT) mechanism use equation (3), this results by performing FFT on equation (1) and equation (2), to convert time domain EEG data into its corresponding frequency domain EEG data.

$$\begin{matrix}{X}_{k} = {\sum }_{n = 0}^{N - 1} {x}_{n} \cdot {c}^{- i2 π kn \text{/} N} & ( 1 ) \\ {X}_{k} = {\sum }_{n = 0}^{N - 1} {x}_{n} \cdot {c}^{- i2 π kn \text{/} N} & ( 2 )\end{matrix}$$

As shown above, the multidimensional DFT transforms an array xₙ with a d-dimensional vector of indices n=(n₁, n₂, . . . n_{d}) by a set of d nested summations (over nⱼ=0 . . . Nⱼ₋₁ for each j), where the division n/N, defined as n/N=(n₁/N₁, n₂/N₂, . . . , n_{d}/N_{d}), is performed element-wise. Further, performing dual parallel FFT on the equation (1) and the equation (2), results equation (3)

$$\frac{N}{{N}_{1}} O ( {N}_{1} log {N}_{1} ) + ⋯ + \frac{N}{{N}_{d}} O ( {N}_{d } log {N}_{d} ) = O ( N [ log {N}_{1} + ⋯ + log {N}_{d} ] ) = O ( N log N ) . \qquad ( 3 )$$

At step 308, the frequency domain EEG data are inputted to a mixer. The mixer shift signals from one frequency range to another. For example, consider a scenario where two signals at frequencies f1 and f2 are applied to a mixer along with the signal from a local oscillator, the mixer produces new signals at the sum f1+f2 and difference f1−f2 of the original frequencies. The mixer produces a frequency shifted EEG data from the inputted frequency domain EEG data. The output signal of mixer is inputted to a low pass filter.

At step 310, the low pass filter having a cutoff frequency, for example 44 Hz, passes the EEG data with a frequency lower than the cutoff frequency and attenuates data with frequencies higher than the cutoff frequency. The output of the low pass filter (denoted as “A”) is inputted to a moving averages filter.

As shown, at step 312, the moving averages filter use equation (4) while considering a slew rate (for example, 2πfv) for smoothing an array of sampled data (EEG data from the low pass filter). The moving average filter is a simple Low Pass FIR (Finite Impulse Response) filter commonly used for smoothing an array of sampled data. In an example, the moving average filter takes M samples of input at a time and performs the average of those M-samples and produces a single output point. The moving average filter eliminates unwanted noisy component (e.g., movement artifact) from the intended data (EEG data).

$$MA ( v ) := {\sum }_{x ∈ V} {p}_{v} ( x ) \cdot f ( x ) \qquad ( 4 )$$

At step 314, the EEG data from the moving average filter is compared with the data of Table 1, to determine the extent of concussion based on the EEG data. Further, the computing device 106 is configured to cause to display data related to the extent of concussion.

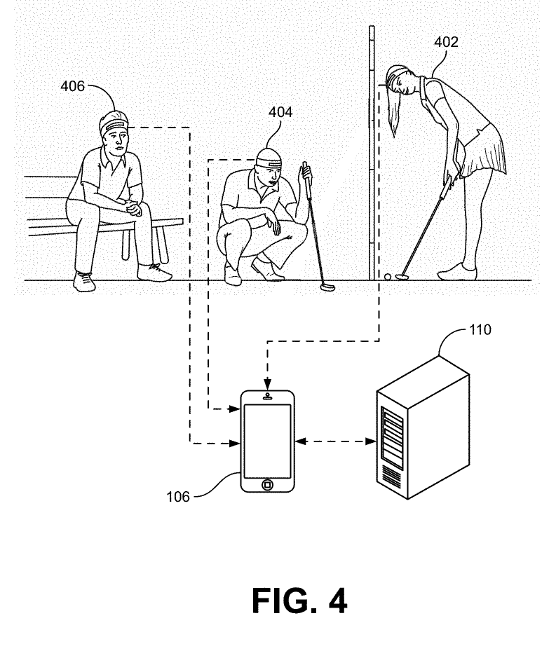

Referring to FIG. 4 is an example scenario illustrating a method for determining the performance metric of the user 104 in a group of users. For example, consider a scenario where a coach 406 is guiding a golfer 402 to play golf in a better way, and a caddie 404 is observing the guiding activities. Each of the person (the coach 406, the golfer 402, or the caddie 404) wearing an external device 102 on their head. In context of the present scenario, the external device 102 is a headband worn by the golfer 402, the caddie 404 and the coach 406. The external device 102 collects EEG data from the Golfer 402, the caddie 404 and the coach 406 and sends the collected data to the computing device 106. It should be understood by those skilled in the art that the external device 102 is capable of capturing EEG data from the golfer 402, the caddie 404 and the coach 406 simultaneously in real time regardless of whether the golfer 402 is in motion and the coach 406 is sited at stationary position at a place watching the golfer 402 play. The computing device 106 converts the received analogue EEG data to digital EEG data. Further, the computing device 106 is configured to eliminate the interference noise from the digital EEG data. In an embodiment, the computing device 106 eliminates the interference noise from the digital EEG data using dual parallel fast Fourier transformation (FFT) process. The dual parallel fast Fourier transformation (FFT) process nullifies the interference that may occurs during the processing of the EEG data from the golfer 402, the caddie 404 and the coach 406.

Further, the computing device 106 is configured to determine the brain state of each user (i.e., the coach 406, the golfer 402 or the caddie 404) by comparing the EEG data of each user with the metric. The brain state represents the psychological parameters of the golfer 402, the caddie 404 and the coach 406. In context of the present scenario, consider desirable brain state is mushin state and desirable objective is winning the match. If the brain state of the user, for example the caddie 404, is not matching with the mushin state frequency, then the computing device 106 is configured to trigger a notification for the caddie 404. In an embodiment, the notification may be a voice message. In an example, the voice message includes step by step guidelines for improving the brain state to achieve the mushin state. The caddie 404 is then required to follow the guidelines in order to coincide with the thoughts of the golfer 402 and the coach 406.

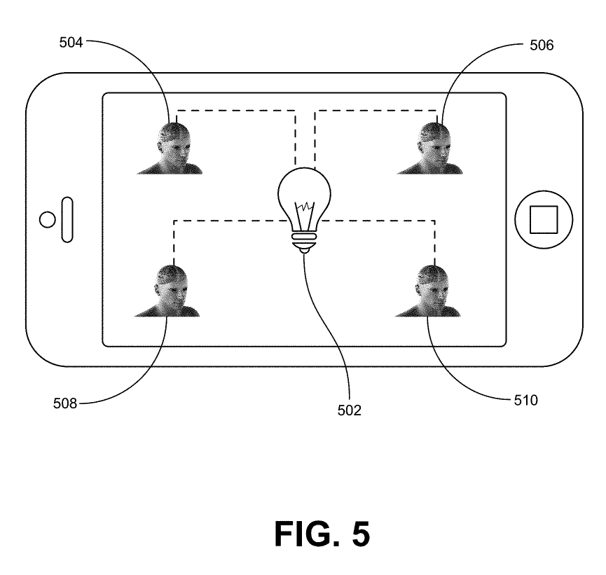

Referring to FIG. 5 is an example illustrating a method for displaying data related to performance metric of the user 104. As shown in FIG. 5, the graphical representation of multiple users 504, 506, 508, 510 is displayed on the computing device 106. For example, consider a scenario, where each of the users 504, 506, 508, 510 wear the external device (eg. headband) 102 on their head. The external devices 102 collect the EEG data from the users 504, 506, 508, 510 and send the data to the computing device 106. It should be understood by those skilled in the art that the capturing means is capable of capturing EEG data from the multiple users 504, 506, 508, 510 simultaneously in real time regardless of whether the users 504, 506, 508, 510 are in motion or sitting idle. The computing device 106 is configured to convert the received analogue EEG data to the digital EEG data. Further, the computing device 106 is configured to eliminate the interference noise from the received digital EEG data. In an embodiment, the computing device 106 removes interference noise from the digital EEG data using dual parallel fast Fourier transformation (FFT) process. The dual parallel fast Fourier transformation (FFT) process nullifies the interference that may occurs while processing EEG data from the multiple users 504, 506, 508, 510.

Further, the computing device 106 is configured to determine the brain state of each user by comparing the EEG data of each user with the metric. In context of the present scenario, consider desirable brain state to be mushin state. If the brain state of the user person (Eg, the user 504) is not matching with the mushin state frequency, then the computing device 106 is configured to trigger a notification for the user 504. In context of the exemplary scenario, the notification may be a graphical element 502. As shown in FIG. 5, the graphical element 502 is a bulb icon. When the multiple people 504, 506, 508, 510 are in the same state, for example, the mushin state, the graphical element 502 is configured to change its display properties. Examples of display properties include, but are not limited to brightness, contrast, color, shape, and size. When the multiple user 504, 506, 508, 510 are in different psychological state, the graphical element 502 remains unchanged. Similarly, when the user, for example the user 504, attains the mushin state, then the connection line between the person 504 and the bulb changes its display properties.

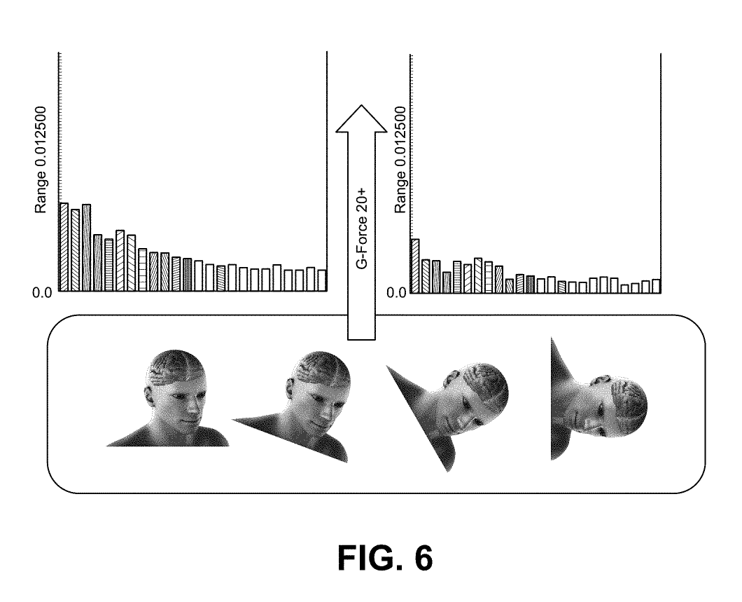

Referring to FIG. 6 is an example illustrating a method for displaying data related to the extent of concussion experienced by the user 104. In an embodiment, the computing device 106 is configured to apply a marker (e.g., label) to the streaming EEG data when a threshold for concussion (G-force) is reached. A noticeable change in the EEG data is displayed on the computing device 106. In an embodiment, the threshold may be different for youth football as compared to professional football, or by gender, age, height, weight, or position. In another embodiment, the threshold may be preprogrammed based on the needs of the user for the activity in which the user will be engaged. In a specific embodiment, acceleration in excess of a ±1 g and sampled rate of 25 Hz may be set as the activity trigger for a walking activity. In another example, the threshold may be a cumulative number that result by adding the markers. The computing device 106 is configured to add a number of data points that are marked as shown in FIG. 6. Consider a scenario, where the threshold for deceleration is 20 marker points. When the addition of the number of data points is equal to 20 then, the computing device 106 is triggered to display the data on the computing device 106.

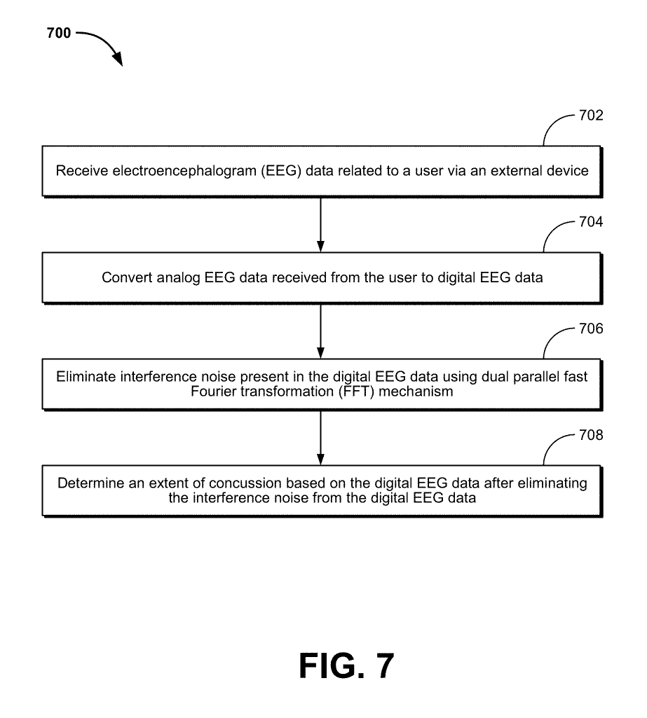

Referring to FIG. 7 is a flow diagram 700 illustrating a method for determining the extent of concussion experienced by the user. At step 702, the method includes receiving EEG data related to the user 104 via the external device 102 associated with the user 104. The method allows the transceiver 208 to receive EEG data related to the user 104 from the external device 102 associated therewith. At step 704, the method includes a step of converting the analog EEG data received from the user 104 to digital EEG data. The method allows the processor 204 to convert the analog EEG data received from the user 104 via the external device 102 to digital EEG data.

At step 706, the method includes a step of eliminating the interference noise from the digital EEG data using a dual parallel fast Fourier transformation (FFT) mechanism. The method allows the G-force analyzer 202 to eliminate the interference noise from the digital EEG data using the dual parallel fast Fourier transformation (FFT) mechanism. At step 708, the method includes describes determining the extent of concussion based on the digital EEG data after eliminating the interference noise from the digital EEG data. The method allows the G-force analyzer 202 to determine the extent of concussion based on the digital EEG data after eliminating the interference noise from the digital EEG data.

The various actions, acts, blocks, steps, or the like in the flow diagram 700 may be performed in the order presented, in a different order or simultaneously. Further, in some embodiments, some of the actions, acts, blocks, steps, or the like may be omitted, added, modified, skipped, or the like without departing from the scope of the invention.

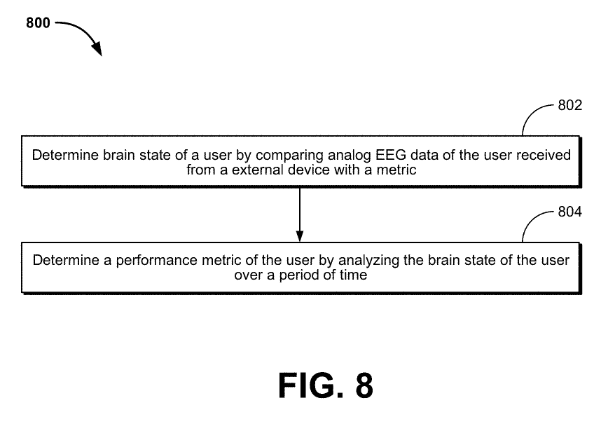

Referring to FIG. 8 is a flow diagram 800 illustrating a method for determining the performance metric of the user 104. At step 802, the method determines a brain state of the user 104 by comparing analog EEG data related to the user 104 received from an external device 102 with a metric. The method allows the processor 204 to determine the brain's state of the user 104 by comparing the analog EEG data of the user 104 with the metric.

At step 804, the method determines the performance metric of the user 104 by analyzing the brain state of the user 104 over a period of time. The method allows the processor 204 to determine the performance metric of the user 104 by analyzing the brain state of the person 104 over a period of time.

The various actions, acts, blocks, steps, or the like in the flow diagram 800 may be performed in the order presented, in a different order or simultaneously. Further, in some embodiments, some of the actions, acts, blocks, steps, or the like may be omitted, added, modified, skipped, or the like without departing from the scope of the invention.

Further to above described system and methodology to determine the extent of concussion experienced by the user 104 and notifying the user 104 of various ways to attain the mushin state in order to succeed in attaining the optimum performance during any athletic activities. Present invention further enables the user 104 in developing technical skills of the game or activities the user 104 is a part of.

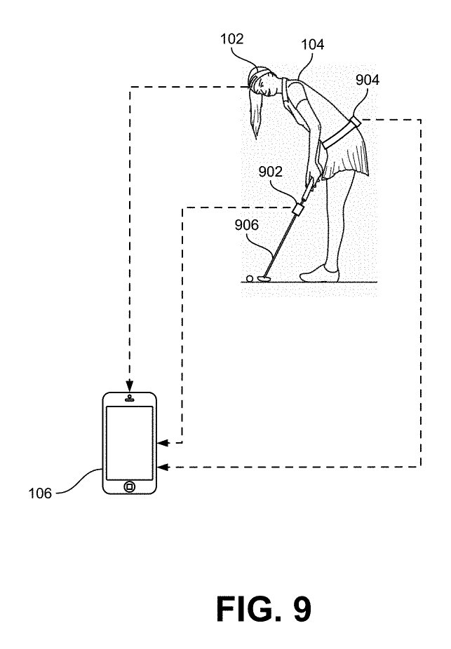

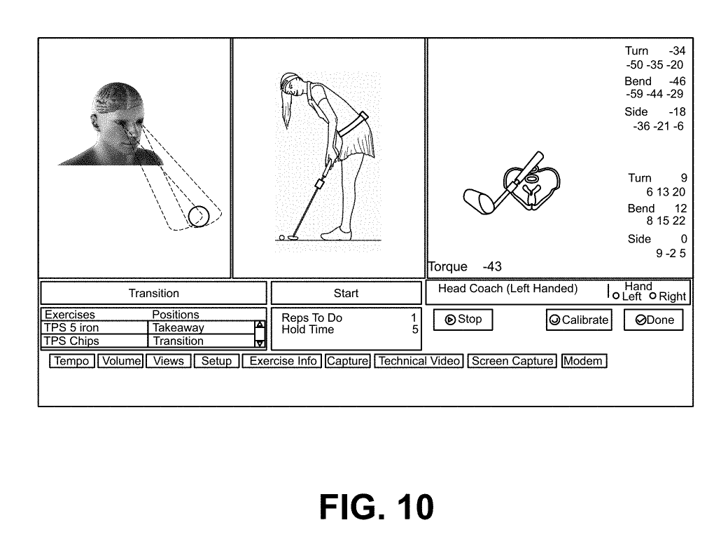

Referring to FIGS. 9-10 which shows diagrams for training the user 104 to develop technical skills related to the athletic activities, and help the user visualize the concussion experienced by him during the training. Although the example illustrated in FIGS. 9-10 are with respect to the game of Golf, it should be understood that the application of the proposed invention is not limited to any specific athletic activities or games. As shown, the external device 102 may be donned by the user 104 during the training. Likewise, one or more sensors, preferably 3D motion sensors such as an accelerometer 902, an tilt sensor 904 may be worn or put on different body locations or onto the golf club 906 held by the user 104 undergoing the training. Although only two sensors 902,904 are shown in use, the accelerometer 902 placed on the club 906 of the user 104, and the tilt sensor 904 worn onto the pelvic area of the user 104, it should be understood that more number of such sensors may be used simultaneously during the training.

In the course of operation, as described above with reference to FIG. 1, the external device 102 collects EEG data from the golfer/user 104 and sends the collected EEG data to the computing device 106 associated with the golfer/user 104 for further processing and determining the concussion and impact of concussion experienced by the user 104 at an instant of time. But as shown in FIG. 9, the computing device 106 may further be configured to receive data from the accelerometer 902 present on the club 906 of the user and the tilt sensor 904 present onto the pelvic area of the user 104 apart from receiving EEG data from the external device 102.

In the example training scenario depicted by FIG. 9-10, the computing device 106 would received the EEG data from the external device 102 and determine the brain state of the golfer/user 104 by comparing the EEG data with the metric. If the determined brain state of the golfer/user 104 is not in the range of the Mushin state frequency, then the computing device 106 provides the training methodology to improve the brain state of the golfer/user 104.

According to the example training scenario shown in FIG. 10, each the brain state of the golfer/user 104 is displayed in different color. For example, if the golfer 104 is in anxiety state, then color of left side of brain is red and color of right side of brain is grey. Similarly, if the golfer 104 is in the Mushin state, then color of left side of the brain is green and color of right side of the brain is blue.

As shown in the example visual screen, the user 104 selects or is given (by his trainer) a specific exercise/drill or set of exercises or drills to practice for example, TPS chip, TPS 5 iron etc. During training, the golfer/user 104 is required to practice or learn specific positions, for example, address takeaway, transition etc. When the user/golfer 104 is taking required position, it is very important to know the body positions of the golfer 104 along with his state of brain. When the user's 104 body position is correct, and he is correctly aiming the golf ball in the mushin state then only the user/golfer 104 succeeds in practicing specific positions of the exercises.

As assistance to the user/golfer 104 undergoing training, the computing device 106 displays the data related to the different sensors (i.e., the accelerometer 902 and the tilt sensor 904) and provide one of audio feedback, visual feedback, or tactile feedback to the golfer 104 to help him align his body position, and his aim at ball with the club. According to the embodiment, the feedback may includes data related to bending, or turning the body position or club by certain degree or angle till the user 104 gets the correct position under the assigned exercise in the mushin state.

Further, as shown, during the training, when the user 104 attains the mushin state with right body position and right position of the club 906 to hit the ball, the computing device 106 would instruct the user/golfer 104 to hold that right position for some specific duration of set time, say 4 sec or 5 sec, which may help the golfer 104 understand or realize the correct position that he achieves during the practice and help him utilize the same during actual sports.

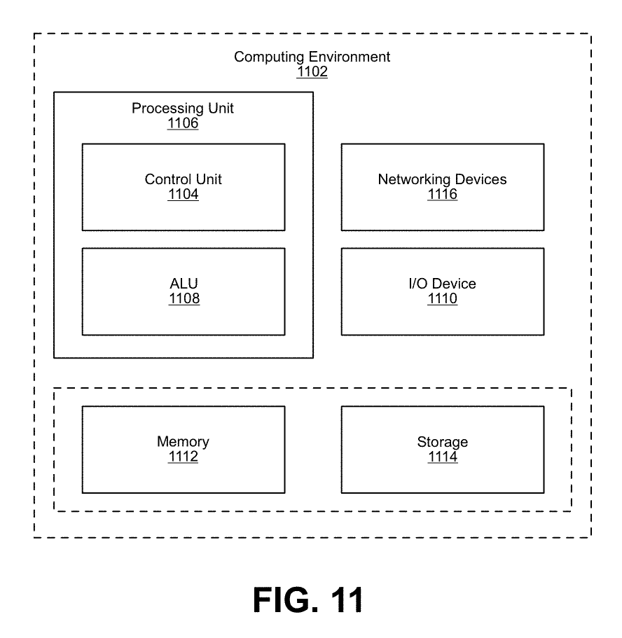

Referring to FIG. 11 illustrates an example computing environment adapted for implementation of the method and system adapted for determining the extent of concussion experienced by the user. As depicted in the FIG. 11, the computing environment 1102 comprises at least one processing unit 1106 that is equipped with a control unit 1104 and an Arithmetic Logic Unit (ALU) 1108, a memory 1112, a storage unit 1114, a plurality of networking devices 1116 and a plurality of input output (110) devices 1110. The processing unit 1106 is responsible for processing the instructions of the schemes. The processing unit 1106 receives commands from the control unit 1104 in order to perform its processing. Further, any logical and arithmetic operations involved in the execution of the instructions are computed with the help of the ALU 1108.

The overall computing environment 1102 can be composed of multiple homogeneous or heterogeneous cores, multiple CPUs of different kinds, special media and other accelerators. The processing unit 1106 is responsible for processing the instructions of the schemes. Further, the plurality of processing units 1106 may be located on a single chip or over multiple chips.

The scheme comprising of instructions and codes required for the implementation are stored in either the memory unit 1112 or the storage 1114 or both. At the time of execution, the instructions may be fetched from the corresponding memory 1112 or storage 1114, and executed by the processing unit 1106.

Further, storage 1114 may include non-volatile storage elements. Examples of such non-volatile storage elements may include magnetic hard discs, optical discs, floppy discs, flash memories, or forms of electrically programmable memories (EPROM) or electrically erasable and programmable (EEPROM) memories. In addition, the storage 1114 may, in some examples, be considered a non-transitory storage medium. The term “non-transitory” may indicate that the storage medium is not embodied in a carrier wave or a propagated signal. However, the term “non-transitory” should not be interpreted that the storage 1114 is non-movable. In some examples, the storage 1114 can be configured to store larger amounts of information than the memory. In certain examples, a non-transitory storage medium may store data that can, over time, change (e.g., in Random Access Memory (RAM) or cache).

In case of any hardware implementations various networking devices 1116 or external I/O devices 1110 may be connected to the computing environment to support the implementation through the networking unit and the I/O device unit.

The embodiments disclosed herein can be implemented through at least one software program running on at least one hardware device and performing network management functions to control the elements. The elements shown in the FIGS. 1 through 11 include blocks which can be at least one of a hardware device, or a combination of hardware device and software units.

The foregoing description of the specific embodiments fully reveals the general nature of the embodiments herein that others can, by applying current knowledge, readily modify or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

What is claimed is:

1. A method for determining an extent of concussion experienced by a user, the method comprising:
receiving, by a computing device, a plurality of electroencephalogram (EEG) data related to the user via an external device associated with the user;
determining, by the computing device, the extent of concussion based on the plurality of EEG data received from the external device associated with the user; and
displaying, by the computing device, a set of data related to the extent of concussion.

2. The method of claim 1, wherein the plurality of EEG data related to the user includes a set of EEG data related to the user before the user is concussed, a set of EEG data related to the user during the time of concussion, and a set of EEG data related to the user after the user is concussed.

3. The method of claim 1, wherein the plurality of EEG data related to the user received from the external device is an analog data.

4. The method of claim 1, wherein the determination of the extent of concussion based on the plurality of EEG data further comprises:
converting, the analog EEG data received from the external device related to the user to digital EEG data;
eliminating, an interference noise from the digital EEG data using a dual parallel fast Fourier transformation (FFT) mechanism; and
determining, the extent of concussion based on the digital EEG data after eliminating the interference noise from the digital EEG data.

5. The method of claim 1 further comprising determining brain's state of the user by comparing the analog EEG data related to the user with a metric.

6. The method of claim 5 further comprising determining a performance metric of the user by analyzing the brain state of the user over a period of time.

7. A computing device for determining an extent of concussion experienced by a user, the computing device comprising:
a memory;
a transceiver to receive a plurality of electroencephalogram (EEG) data related to the user via an external device associated with the user;
a processor coupled to the memory;
a G-force analyzer communicatively coupled to the processor, the processor configured to:
determine the extent of concussion based on the plurality of EEG data; and
display a set of data related to the extent of concussion.

8. The computing device of claim 7, wherein the processor is configured to determine the extent of concussion based on the plurality of EEG data related to the user's brain activity before the user is concussed, during the time of concussion, and after the user is concussed.

9. The computing device of claim 7, wherein the plurality of EEG data related to the user received from the external device is an analog data.

10. The computing device of claim 7, wherein the processor is configured to determine the extent of concussion based on the plurality of EEG data received from the user via the external device, the determination is performed by the processor by:
converting, the analog EEG data related to the user to digital EEG data;
eliminating, an interference noise from the digital EEG data using a dual parallel fast Fourier transformation (FFT) mechanism; and
determining, the extent of concussion based on the digital EEG data after eliminating the interference noise from the digital EEG data.

11. The computing device of claim 7, wherein the processor is further configured to determine brain state of the user by comparing the analog EEG data related to the user with a metric.

12. The computing device of claim 7, wherein the processor is further configured to:
determine a performance metric of the user by analyzing the brain state of the user over a period of time; and
display data related to the performance metric.

13. A computer program product comprising a computer executable program code recorded on a computer readable non-transitory storage medium, the computer executable program code when executed causing the computer to perform the following steps:
receive a plurality of electroencephalogram (EEG) data related to the user via an external device associated with the user;
determine the extent of concussion based on the plurality of EEG data received from the user via the external device; and
display a set of data related to the extent of concussion.
